(19) United States
(12) Patent Application Publication
(10) Pub. No.: US 2007/0010823 A1
(43) Pub. Date: Jan. 11, 2007

(54) Arthroscopic shaver system

(75) Inventor: Theodore R. Kucklick, Los Gatos, CA (US)

Correspondence Address: Crockett & Crockett;Suite 400, 24012 Calle De La Plata, Laguna Hills, CA, 92653, US

(73) Assignee: Cannuflow, Inc.

(21) Appl. No.: 11/180,118

(22) Filed: Jul. 11, 2005

(51) Int. Cl. A61B 17/00

(52) U.S. Cl. 606/80

(57) ABSTRACT

A system for providing aspiration and irrigation during a medical procedure with an arthroscopic shaver.

### FIELD OF THE INVENTION

The inventions described below relate the field of arthroscopic shavers.

### BACKGROUND OF THE INVENTION

Various arthroscopic shavers are used in arthroscopic procedures to remove tissue and reshape a patient's anatomy. A surgeon may use an arthroscopic shaver to remove bone or cartilage and other soft tissue from a patient's joint, or in procedures such as septoplasty (sinus reduction). The shavers in use include a rotating burr housed within a rigid insertion tube but exposed to body tissue through a small aperture in the side or end of the insertion tube. Suction is applied through the insertion tube so that debrided body tissue can be sucked into the tube and removed from the body. This requires efficient irrigation of fluid and aspiration of debris during many surgical procedures. Unwanted tissue may also be removed with manual instruments such as a punch or energy delivering instruments such as a radiofrequency powered device, or a laser.

Currently available arthroscopic shaver devices tend to clog quickly with surgical debris. Some resected tissue is tough and stringy, and gets wrapped around the cutting burr. Some procedures produce an amount of debrided tissue that overwhelms the aspiration capabilities of the system. In these situations, the arthroscopic shaver may have to be removed, cleaned, and re-inserted into the surgical field repeatedly during the course of a single procedure.

### SUMMARY

The devices and methods described below provide for enhanced aspiration and clearing of debris from a surgical field during use of an arthroscopic shaver. An arthroscopic shaver is provided with a sheath having one or more lumens for irrigating or aspirating a surgical field, with apertures disposed just proximal to the cutting burr of the shaver. Associated sources of pressurized irrigation fluid and vacuum are provided in fluid communication with the lumen(s) of the sheath and typical suction lumen of the arthroscopic shaver, and control valves for aligning, at the option of surgeon using the device, to the pressure or vacuum sources. Additionally, valves controlling pressurized irrigant supply to the tip of the catheter are controlled by hydraulic, mechanical, or electromechanical interlock to coordinate irrigant supply with rotation of the cutter.

### BRIEF DESCRIPTION OF THE DRAWINGS

FIG. 1 illustrates an arthroscopic shaver with an inflow/outflow sheath disposed over the shaver.

FIG. 2 shows a cross section of the system at the apertures of the inflow/outflow sheath.

### DETAILED DESCRIPTION OF THE INVENTION

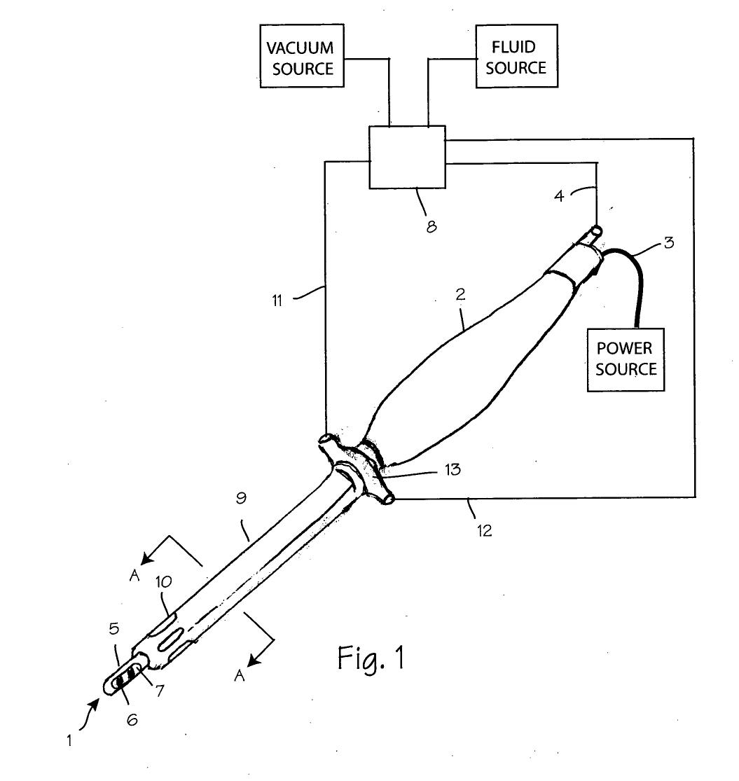

FIG. 1 illustrates an arthroscopic shaver with an inflow/outflow sheath disposed over the shaver. The arthroscopic shaver 1 comprises a handle 2 with a motor and power cord 3, vacuum/irrigation line 4 and a rigid insertion tube 5. Inside the rigid insertion tube, the cutting element 6, which may be any burr, shaver, or cutter design, is mounted on the tip of a rotary shaft which is connected to the motor in the handle. A small aperture 7 in the insertion tube, typically side-facing as shown, exposes the cutting element to body tissue in the surgical field. The vacuum/irrigation line 4 in fluid communication with the luminal space within the insertion tube is provided with a control valve 8 for selective applying suction or irrigation fluid to the shaver aperture, to aspirate the surgical space or flush the burr at the option of the surgeon. For endoscopic applications, the insertion tube and rotary shaft may be flexible, and the motor may be located at some distance proximal to the handle. The inflow/outflow sheath 9 is shown disposed over the insertion tube of the shaver. The sheath is sized relative to the insertion tube, or secured relative to the insertion tube, such that the distal end of the sheath is just proximal to the aperture of the shaver. The sheath may be rigid (i.e., it may be a cannula) for use in arthroscopic procedures, or the sheath may be flexible for use in laparoscopic and other procedures. At the distal end of the sheath, one or more sheath apertures 10 (either side facing, through the outer side wall of the sheath, or distally facing through the distal tip of the sheath) provide for fluid communication from irrigation and/or vacuum sources, lumens formed in the sheath, and the surgical field. A first sheath vacuum/irrigation line 11, and an additional sheath vacuum/irrigation line 12 fitted in fluid communication with the lumens of the sheath through sheath fluid coupling 13. Attached to each vacuum/irrigation line is a control valve 8 for selectively applying suction or irrigation fluid to the shaver aperture, to aspirate the surgical space or flush the burr at the option of the surgeon, and of course suitable sources of vacuum and irrigation fluid. The control valve 8 supplying the sheath and the insertion tube irrigation flow may be interlocked to prevent flow in unintended situations, and as shown in FIG. 1, a suitable vacuum switch and cut-off valve arrangement senses the vacuum created within the insertion rod by the spinning rotary shaft or motor or other rotating component, and the cutoff valves are opened in response to allow surgeon initiated flow through the control valves.

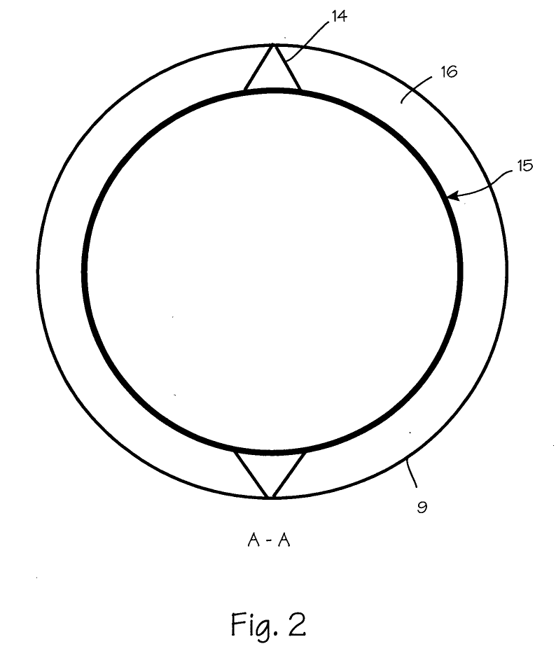

FIG. 2 shows a cross section of the system at the apertures of the inflow/outflow sheath, just proximal to the distal end of the sheath, and the shaver aperture. As illustrated, the inflow/outflow sheath 9 has no inner wall, and depends on the cooperative relation of the longitudinal webs 14 that extend from the inner surface of the sheath and the outer wall 15 of the insertion tube 5 of the shaver to establish substantially fluid tight seals. Thus, the insertion tube, the sheath and the longitudinal webs define outer lumens 16 that permit fluid flow between a fluid or vacuum source and a surgical site. (The inflow/outflow sheath may also have an inner tube, outer tube, and one or more lumens defined between the two tubes.) The apertures of the sheath communicate through the wall of the sheath to permit suction and irrigation.

In use, the surgeon uses the arthroscopic shaver as dictated by the surgery to be accomplished. The surgeon applies vacuum to the shaver insertion tube to aspirate the surgical field. In addition, the surgeon may apply vacuum to the sheath lumens to provide additional aspiration, or provide additional irrigation to the surgical field. Should the shaver become clogged, the surgeon may direct irrigation flow through the shaver lumen to back flush the burr into the surgical field and then aspirate the field through the sheath lumens. The surgeon may operate the several lumens to aspirate or irrigate as the contingencies of any particular surgery require. Should the sheath become clogged, it is a simple matter to slide the sheath off of the shaver and clear the debris, replace it on the shaver cannula shaft and resume surgery.

The sheath and irrigation/aspiration system may also be fitted to other tools, including cautery tools, radiofrequency ablation device, a surgical laser, or thermal device, cutter of various types, and manual surgical debridement tool such as a cutter, rasp, rongeur, punch, or scissors.

While the preferred embodiments of the devices and methods have been described in reference to the environment in which they were developed, they are merely illustrative of the principles of the inventions. Other embodiments and configurations may be devised without departing from the spirit of the inventions and the scope of the appended claims.

We claim:

1. A system for providing aspiration and irrigation during a medical procedure with an arthroscopic shaver, said system comprising:
an arthroscopic shaver suitable for performing an arthroscopic surgical procedure comprising an insertion tube with a shaver aperture and a cutting element disposed within the insertion tube; and
a sheath having a distal end, a proximal end and an inner diameter sized and dimensioned to permit fluid flow between an inner surface of the sheath and an outer surface of the insertion tube when the shaver is disposed within the sheath, said sheath further having a plurality of webs extending inwardly from the inner surface of said sheath and running longitudinally along said sheath;
wherein the webs define outer lumens between the outer surface of the insertion tube and the inner surface of the sheath;
wherein the sheath is adapted to be removably disposed over the shaver.

2. The system of claim 1 wherein the distal end of the sheath further comprises one or more sheath apertures in fluid communication with one or more outer lumens.

3. The system of claim 2 wherein a distal end of the shaver extends distally from the distal end of the sheath when the sheath is disposed over the insertion tube of the shaver.

4. The system of claim 2 further comprising a fluid coupling disposed on the proximal end of the sheath in fluid communication with one or more outer lumens.

5. The system of claim 4 further comprising a fluid source in fluid communication with the coupling.

6. The system of claim 4 further comprising a vacuum source in fluid communication with the coupling.

7. The system of claim 4 further comprising a control valve in fluid communication with the fluid source, the vacuum source, the fluid coupling and the insertion tube, said control valve adapted to selectively supply fluid or vacuum to the fluid coupling and the insertion tube.

8. The system of claim 7 wherein the control valve may be interlocked to prevent fluid flow during unintended situations.

9. The system of claim 1 wherein the distal end of the sheath is flexible.
